(19) United States
(12) Patent Application Publication
(10) Pub. No.: US 2011/0257991 A1
(43) Pub. Date: Oct. 20, 2011

(54) Pharmacy Product Inventory Control or Redistribution

(76) Inventor: Anand Shukla, WestMinster, CO (US)

(21) Appl. No.: 13/015,060

(22) Filed: Jan. 27, 2011

Related U.S. Application Data
(60) Provisional application No. 61/307,763, filed on Feb. 24, 2010.

(51) Int. Cl. G06Q 50/00; G06Q 10/00

(52) U.S. Cl. 705/2

(57) ABSTRACT

A method to reduce or eliminate the generation of expired products and manage pharmacy inventories. The method includes maintaining an online pharmacy inventory database among a plurality of participating network pharmacies, identifying over-stock products, non-moving products, slow moving products, and un-wanted products from the plurality of participating network pharmacies, and generating a redistribution list of one or more products.

### CROSS-REFERENCE TO RELATED APPLICATIONS

This application claims priority from a U.S. provisional patent application, Ser. No. 61/307,763, filed on Feb. 24, 2010, and entitled “Pharmacy Drug Inventory Control,” which is hereby incorporated by reference.

### BACKGROUND OF THE INVENTION

1. Field of the Invention

This invention relates generally to a method and system to reduce the generation of expired products. More specifically, the present invention relates to inventory management and inter-pharmacy transfer of products.

2. Description of the Prior Art

Several million Americans cannot afford prescription drugs because of rising drug prices, at the same time expensive prescription drugs are wasted every day. The projected value of all prescription products returned in the Unites States for which manufacturer credit is requested is estimated to range from $2.6 to 4.2 billion, according to the HDMA Factbook for 2008-2009.

A typical pharmacy currently rejects approximately 3% to 10% of the entire store inventory every year. Poor inventory management is a major factor responsible for the high rate of rejects. Pharmacy operators typically receive only 30% to 50% of the value on their returned goods. This is because the other 50% to 70% goes toward reverse distributor commissions, non-returnable goods, disposal costs, and other logistical expenses. As a result of heavy losses due to expired drugs and with constantly changing pharmacy practices and tougher laws, many independent pharmacies are on the verge of going out of business.

Chain pharmacies, on the other hand, are managed by corporations. Inventories for most of the chain store pharmacies are controlled by their central purchasing location. Some chain pharmacies use inventory management software to manage their store inventories, but the number of expired drugs generated from the chain pharmacies is still very high compared to independent pharmacies.

Hospital pharmacies also have very high rate of drug returns. In general, the types of drugs that expire in hospitals are comparatively more expensive compared to the drugs carried by chain and independent pharmacies. This is because hospital pharmacies supply medicines to surgery and emergency departments. Mail-order and long-term pharmacies carry very high volumes of prescription drugs. Expired drugs generated from these pharmacies are usually higher in cost than expired drugs generated by independent pharmacies and chain pharmacies. Again there is a lack of formal communication between the common control hospital pharmacies to tackle unsalable/overstock prescription drugs.

As a result of the factors previously listed, prescription drug waste is increasing at a high rate. According to the HDMA Fact book for 2008-2009, several other factors unrelated to the inventory management are also contributing to the increase in drug waste. These factors include drug recalls, shipping and handling damage to the goods, short shelf life of certain drugs, odd product packaging configurations, sudden changes in the prescription pattern of a particular class of drug, generic competition, changes in Medicaid and Medicare coverage, inconsistent supply of the drug due to lack of interest of doctors, aggression promotion cycles by the manufacturers, and the increasing ineffectiveness of certain classes of drugs, such as antibiotics, against evolving microbes.

The generation of expired products and the loss in revenue is just one problem. Many hours of manpower are lost at the pharmaceutical distribution sites in managing the expired products. The process involves inefficiently recording every count of pills either by hand or some form of digital device. Segregating expired products from the non-expired products, labeling, handling, shipping, and processing are other challenges. At a time when pharmacies are struggling to keep their doors open by cutting their workforce, wasting extra hours of manpower in managing expired products is unacceptable.

In the United States, several thousand drug manufacturers have close to 50,000 drug products in the distribution channels. Each manufacturer has a unique drug return policy. For example, some manufacturers do not grant any credit for expired drugs. As a result, once a drug is sold to the pharmacy it cannot be returned. Some manufacturers grant only partial credit on returned drugs. The amount of the partial credit typically ranges anywhere from 30% to 95%.

Few manufacturers do not grant any credit on partially filled bottles. In some cases, partial returns are accepted, but only if a pharmacy meets all the partial returns criteria set by the manufacturer. Often the pharmacies do not meet the partial credit criteria. It has become difficult for the pharmacy operators to know how much credit they will receive for their returned goods and when the credit will arrive. In addition, credit from the wholesaler or manufacturer does not come for months. Sometimes, one manufacturer has different return polices for different products.

Pharmacy operators are unable to keep track of hundreds of thousands of constantly changing drug return policies. In the end, pharmacy operators end up recovering only approximately 30% to 50% of the original purchase price on expired and over stock items. Expired products in the pharmacy have both financial and safety concerns. Therefore, early detection of each expired product is critical because most of the manufacturers do not provide credit on products beyond three to six months after the expiration date. Currently, there is not a single inventory system that allows a pharmacy operator to know which product in their pharmacy has expired or will expire in the near future. Often pharmacy operators will miss the manufacturers' post expiration window to return the expired products to qualify for credit from the manufacturer.

Many of the expired products are non-moving or slow moving products. This is particularly true for the owners of multiple pharmacies. They do not have a central database where they can view their daily sales transactions, purchases, and returned products. They could make better decisions if they had the visibility, accountability, and control over their store inventories. An online inventory database along with the statistical data on their top sellers, non-movers and slow-movers would provide them with early warning indications on particular products. Based on this information, they could make more informed decisions, such as what to order, how much to order, what to return, when to return, and how much credit they should expect.

Although inter-pharmacy transfers are legally allowed between commonly controlled pharmacies, transfers are sporadic and expensive products go to reject. Few states allow one pharmacy to transfer their overstock prescription drugs to other pharmacy within the state. Inter-pharmacy resales of prescription drugs are governed by each individual state board of pharmacy. And rules for the inter-pharmacy transfer of prescription drugs vary from state to state. A few states allow pharmacies to resell up to 5% of their annual sales of prescription drugs. At least one state allows only the products in the original container, but partial prescription drugs are not allowed. In reality, independent pharmacies, chain pharmacies, and hospital pharmacies rely on informal personal networks that result in the generation of prescription drug waste and the expenditure of many manpower hours. The lack of a single source of visibility and accountability leads to inefficient and needless loss of expensive medicine.

### SUMMARY OF THE INVENTION

The present invention provides a method and system to improve product inventory management, reduce the amount of expired product generated in the health care sector, and resale/redistribution of prescription products between prescription products outlets. The invention can be implemented in numerous ways, such as by a method, a computer network, embedded applications, or a program stored on computer readable media. Other implementations, although not listed here, are included in this application. Two aspects of the invention are described below.

A first aspect of the invention is directed to a method to manage pharmacy inventory, including: maintaining an online pharmacy inventory among a plurality of participating network pharmacies by using one or more specifically programmed data processing systems, identifying over-stock products, non-moving products, slow moving products, un-wanted products, and near expiry or expired products from the plurality of participating network pharmacies, generating a redistribution list of one or more products, and matching product supply and demand between the plurality of participating network pharmacies.

A second aspect of the invention is directed to a system to manage pharmacy inventory, including: a data processing system network to maintain an online pharmacy inventory among a plurality of participating network pharmacies, using one or more specifically programmed data processing systems, a database on the data processing system to identify over-stock products, non-moving products, slow moving products, and un-wanted products from the plurality of participating network pharmacies, a program module on the data processing system to generate a redistribution list of one or more products, and a program module to match product supply and demand between the plurality of participating network pharmacies.

These and other aspects of the invention will become apparent to those skilled in the art from the following detailed description of the invention and the accompanying drawings.

### BRIEF DESCRIPTION OF THE DRAWINGS

Non-limiting and non-exhaustive embodiments of the invention are described with reference to the following figures.

FIG. 1 illustrates services that are offered, in accordance with one embodiment of the invention.

FIG. 2 illustrates services that are offered, in accordance with one embodiment of the invention.

FIG. 3A illustrates a block diagram of data inputs to a database of an online pharmacy inventory, in accordance with one embodiment of the invention.

FIG. 3B illustrates a block diagram of data inputs to an alternative database of an online pharmacy inventory, in accordance with another embodiment of the invention.

FIG. 4 illustrates a flowchart of a method to reduce product inventories, in accordance with one embodiment of the invention.

FIG. 5 illustrates a flowchart of a method to reduce product inventories, in accordance with one embodiment of the invention.

FIG. 6 illustrates a flowchart of a method to reduce product inventories, in accordance with one embodiment of the invention.

FIG. 7 illustrates a flowchart of a hypothetical and non-limiting product overstock example at a pharmacy.

FIG. 8 illustrates a hypothetical and non-limiting example of a web-site advertisement.

FIG. 9 illustrates a hypothetical and non-limiting example of a database entry, in accordance with one embodiment of the invention.

FIG. 10 illustrates a data processing system to implement the invention, in accordance with one embodiment of the invention.

FIG. 11 illustrates a page of an inventory table of a database, in accordance with one embodiment of the invention.

FIG. 12 illustrates a page of a sales data table of a database, in accordance with one embodiment of the invention.

FIG. 13 illustrates a page of an invoice table of a database, in accordance with one embodiment of the invention.

FIG. 14 illustrates a page of a threshold quantity table of a database, in accordance with one embodiment of the invention.

FIG. 15 illustrates a page of a product expiration table of a database, in accordance with one embodiment of the invention.

FIG. 16 illustrates a database update sequence, in accordance with one embodiment of the invention.

### DETAILED DESCRIPTION OF PREFERRED EMBODIMENTS

In the following description, numerous specific details are set forth such as examples of specific systems, languages, components, etc. in order to provide a thorough understanding of the present invention. It will be apparent, however, to one skilled in the art that these specific details need not be employed to practice the present invention. In other instances, well-known methods have not been described in detail in order to avoid unnecessarily obscuring the present invention. In the discussion of the present invention, pharmacy will be defined to include any licensed premise involved in preparing and dispensing products and medicines (e.g., independent, chain, super market, mail-order pharmacies, long-term care pharmacies, veterinary pharmacies, pet pharmacies, hospital pharmacies, doctor offices containing large bulk of samples, or an equivalent). This invention also extends to wholesaler and pharmaceutical manufacturer warehouses carrying prescription products.

In one embodiment, a computer file is defined to include data that serve as input to a device or program. The types of input files various embodiments of the invention can use include one or more physical inventory files, one or more sales files, one or more purchasing invoices, and one or more returned product invoices, where any of these files can be received in a real time or at predetermined interval. In one embodiment, a physical inventory file can be generated as a one time input and generated every year thereafter. Sales and purchasing files can be updated by real-time, intermittent or transaction-based updates. Returned product invoices can appear randomly, multiple times throughout the year.

The present invention includes various operations, which will be described below. The operations of the present invention could be performed by hardware components or could be embodied in machine-executable instructions, which would be used to cause a special-purpose processor programmed with the instructions to perform the operations. In certain embodiments, the method can be performed by processing logic that could comprise hardware (e.g., dedicated logic, programmable logic, microcode, or an equivalent), software (such as run on a computer system, a specifically programmed machine, or an equivalent data processing system), or a combination of both hardware and software.

The present invention can be provided as a computer program product, or software that can include a machine-readable medium having stored thereon instructions, which can be used to program a computer system (or other equivalent data processing systems or electronic devices) to perform a process according to the present invention. A machine-readable medium includes any mechanism for storing or transmitting information in a form (e.g., software, processing application) readable by a machine (e.g., a computer, or an equivalent data processing system or electronic device). The machine-readable medium could include, but is not limited to, magnetic storage medium (e.g., floppy diskette); optical storage medium (e.g., CD-ROM, DVD, or an equivalent); magneto-optical storage medium; read only memory (ROM); random access memory (RAM); erasable programmable memory (e.g., EPROM, EEPROM, or an equivalent); flash memory; electrical, optical, acoustical or other form of propagated signal (e.g., carrier waves, infrared signals, digital signals, or an equivalent); or other type of medium suitable for storing electronic instructions.

The present invention can also be practiced in distributed computing environments where the machine readable medium is stored on and/or executed by more than one computer system. In addition, the information transferred between computer systems could either be pulled or pushed across the communication medium connecting the computer systems.

Some portions of the description that follow are presented in terms of algorithms and symbolic representations of operations on data bits that can be stored within a memory and operated on by a processor. These algorithmic descriptions and representations are the means used by those skilled in the art to effectively convey their work. An algorithm is generally conceived to be a self-consistent sequence of acts leading to a desired result. The acts are those requiring manipulation of quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, parameters, or equivalent terms. Various embodiments provide methods and systems for maintaining real-time, perpetual, transaction-based, and/or intermittent inventory of pharmaceutical and chemical products for users to identify idle inventory and provide a platform for reselling, redistributing, or transferring products between two or more entities. Various embodiment methods and systems allow users to track the movement of products which can selectively include an expiration date using one more electronic communication systems.

In one embodiment of the invention, a supply-demand algorithm (implemented on a computer) identifies unused products. And a distribution model (using the Internet, a dedicated computer network, a telephone network, or an equivalent) allows resale/redistribution of products by an inter-pharmacy transfer of products, before the products reach their expiration dates. In one embodiment, pharmacy inventory management is taken outside the pharmacy by utilizing an online Windows® based operating system or an alternate operating system based inventory database operated on one or more data processing systems (mainframe computers, client-server network of computers, cloud computing, desktop computers, laptop computers, palm computers, hand-held communications devices, or an equivalent).

In one embodiment, a database tracks the movement of products and identifies non-moving products, slow-moving products, over-stock products, near expiry products, expired products and unwanted products within the inventories of participating pharmacies. These products are then resold and redistributed among participating network pharmacies via a secure online database. This provides an information technology platform and supports the logistics for inter-pharmacy product transfers, without taking physical possession of the products. These inter-pharmacy product transfers make either occur within one state, or between states that legally permit inter-state product transfers between pharmacies.

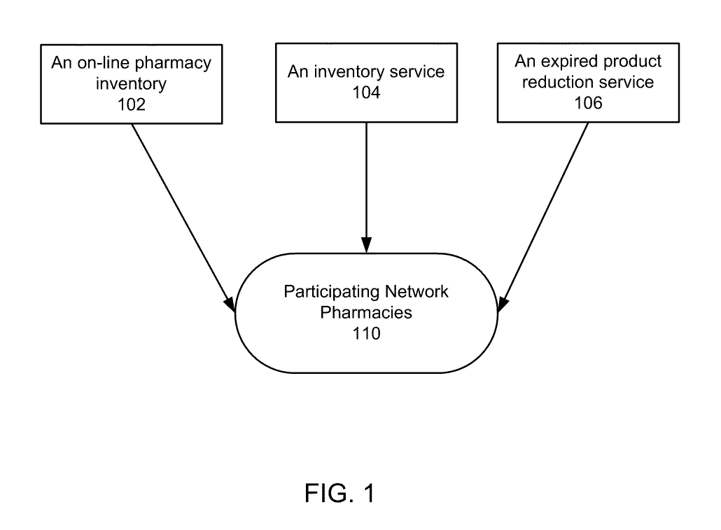

FIG. 1 illustrates services that can be offered, in accordance with one embodiment of the invention. In one embodiment of the invention, several services are offered to participating network pharmacies 110. Such services include an online pharmacy inventory 102; an inventory service 104 in order to provide visibility and accountability; and an expired product reduction service 106 to redistribute over-stock products. In one embodiment of the invention, the online pharmacy inventory 102 is maintained by capturing daily sales and purchasing data via and electronic data interchange interface or other techniques and the current inventory table will provide overview to the pharmacy. In one embodiment of the invention, an inventory service 104 allows a pharmacy to view non-moving items, slow moving items, items owed to patients, near expiry products, expired products, statistical data of prescription sold and creation of custom reports. In one embodiment of the invention, participating network pharmacies can be allowed to put their overstock products, near-expired products, and unwanted products on resale with other participating network pharmacies. In one embodiment of the invention, transferring and receiving pharmacies can be made aware of requests for transfers and tracking of the transfers and invoices and pricing will be provided. In one embodiment of the invention, full logistic support for the inter pharmacy transfers will be provided, and if a shipping company is specifically agreed upon in advance, this will also allow automatic printing of shipping labels, package tracking, and receipt confirmation.

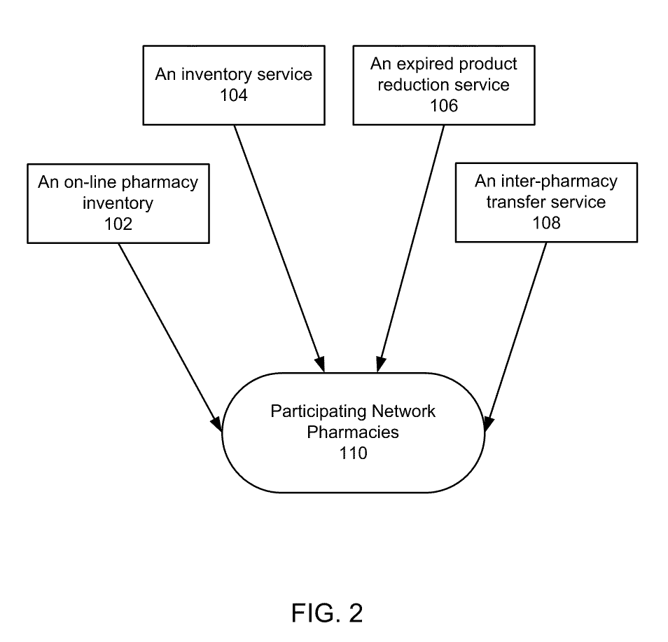

FIG. 2 illustrates services that can be offered, in accordance with one embodiment of the invention. In this embodiment of the invention, one or more of several services are offered to participating network pharmacies 110. Such services include an online pharmacy inventory 102; an inventory service 104 in order to provide visibility and accountability, to increase control, to allow for informed decision-making and ordering, and to facilitate daily tracking; expired product reduction services 106, which includes expired products reduction or elimination (prevention) services in order to reduce manpower management, increase productivity, reduce loss, protect the environment from the toxic waste of expired or unwanted products; and an inter-pharmacy transfer assistance 108 to re-sell and redistribute over-stock products in order to prevent losses due to non-returnable items, avoid stocking hard-to-sell products, and reduce high inventory costs.

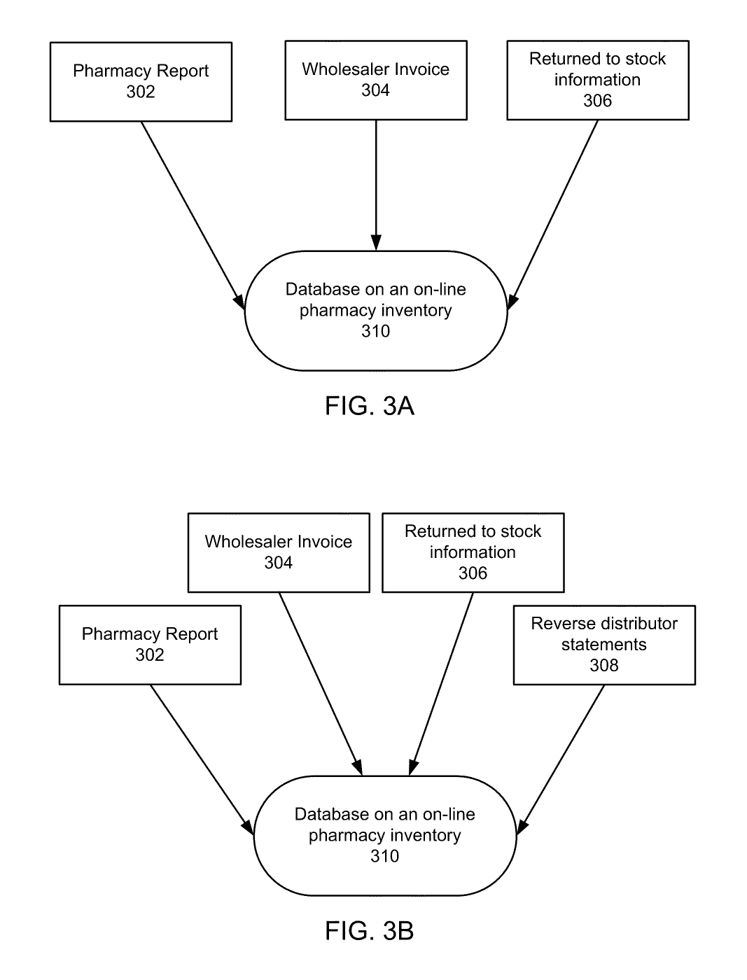

FIG. 3A illustrates a block diagram of data inputs to a database of an online pharmacy inventory, in accordance with one embodiment of the invention. Module 302 includes a pharmacy sales report (e.g., manual reports via email and fax or electronically directly from a switch provider using FTP, EDI, and other non-restricted means). Module 304 includes a wholesaler invoice (some pharmacies use multiple wholesalers). Wholesaler invoices are received via one or more of the following: email, EDI, FTP, fax, logging into a clients account, or an equivalent. Module 306 includes returned to stock information (e.g., returned to stock item is captured either from pharmacy sales report or manually processed by a client on their account web-site). Module 310 is the database of an online pharmacy inventory.

Pharmacy sales reports can be generated using different methods. In one embodiment, the pharmacy sales reports are generated in the pharmacy management system computer at each pharmacy at the end of the day. These reports could consist of a complete transaction history for a particular day. In one embodiment, the pharmacy sales report could be standardized or customized and stored in the pharmacy management system. In one embodiment, if a pharmacy is using multiple computer stations, the pharmacy sales report is stored and retrieved from multiple computers, and the pharmacy data are stored in the pharmacy's local server. In one embodiment, a pharmacy sales report includes the transaction history of pharmacy operation for a specific day which also includes the number of prescriptions filled, the amount of products dispensed per prescription, date of dispensing of each product, and patient information.

In another embodiment, the pharmacy sales reports can be generated by switch providers. Switch providers enable pharmacies to submit claims to third party insurance companies. Data that is transmitted through the switch provider to the insurance companies could be collected and used to generate one or more pharmacy sales reports

In another embodiment, the pharmacy sales reports use barcodes on products. At a point of sale, product barcodes can be scanned and the data can be collected to generate a pharmacy sales report. In another embodiment, a wholesaler invoice can be generated by a wholesaler when a pharmacy places an order with the wholesaler.

In one embodiment, a front-end application allows customers to see data in read-only format from different tables including, Inventory, Sales Data, Invoice, Item Owed, Threshold Qty, Return to Wholesaler, Expiring 1M (products expiring in next 1 month), Expiring 6M (expiring in next 6 months) and Expired (already expired products). In one embodiment, a front-end application also allows customers to do a search from their inventory database.

In one embodiment, a back-end application is used by a programmer to process the different input files (such as inventory, sales, invoice files, returned products) and, to import data to MySQL database. In one embodiment, a developer executes these programs manually or with help of software on daily basis or in real time. In one embodiment, the output of these programs includes several CSV files (e.g., each file representing single table in database), which are then used to update the MySQL database on a server using MySQL's import utility.

In one embodiment, the architecture of the system uses heterogeneous technologies involving PHP front-end processing and Java for back-end processing. The technical environment can be homogeneous, if similar technologies are used for the front-end processing and back-end processing. For example, one embodiment can use Java/J2EE or .NET for the front-end and back-end processing. Homogeneous technologies would eliminate a manual MySQL import utility step and allow the entire process to be automated.

In various embodiments, a web browser (e.g., Internet Explorer, Firefox, Google Chrome, or an equivalent) can be used. In one embodiment, HTML is involved in the front-end web-site development. In one embodiment, a PHP web application hosted on an Apache application server is used for creating the front-end tables. In one embodiment, a user interface is provided for a client to login to their account and view their profile and listing screens that list tables that contains data.

In one embodiment the database and the website can be located on commercially available shared server. In one embodiment, a new web application would make use of a new, stable JDK (e.g., level 1.6 or an equivalent) for development. In one embodiment, the Apache Tomcat 6 is used as an application server for hosting the web application. In one embodiment, the system would continue using same MySQL 5.1 as a back-end database server.

In one embodiment, the web application will be developed using a Struts framework. The Struts framework is based on a Model-View-Controller architecture. It makes development of Java-based web applications relatively easier.

FIG. 3B illustrates a block diagram of data inputs to an alternative database of an online pharmacy inventory, in accordance with another embodiment of the invention. Module 302 includes a pharmacy report. Module 304 includes a wholesaler invoice. Module 306 includes returned to stock information. Module 308 includes reverse distributor statements. Module 310 is the database of an online pharmacy inventory.

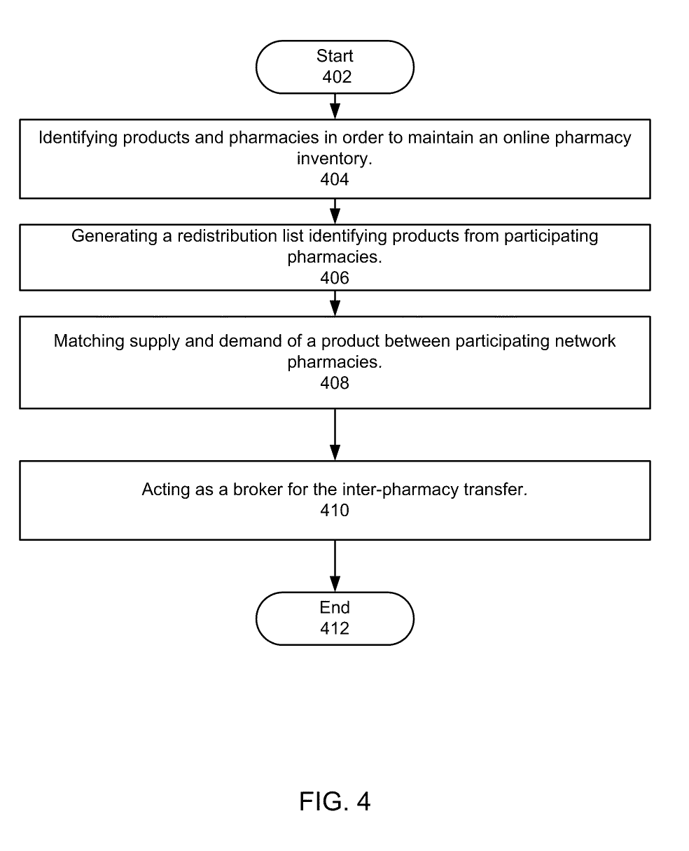

FIG. 4 illustrates a flowchart of a method to reduce product inventories, according to one embodiment of the invention. The sequence starts in operation 402. Operation 404 includes identifying products and pharmacies in order to maintain an online pharmacy inventory. Operation 406 includes generating a redistribution list (e.g., a list of products for resale, a list or products for redistribution, or an equivalent list) identifying over-stock products, non-moving products, slow moving products, and un-wanted products from the participating network pharmacies. Operation 408 includes matching supply and demand of a product between participating network pharmacies (e.g., by a computer online network, or an equivalent). Operation 410 includes acting as a broker for the inter-pharmacy transfer (e.g., of unused/overstock products). The method ends in operation 412.

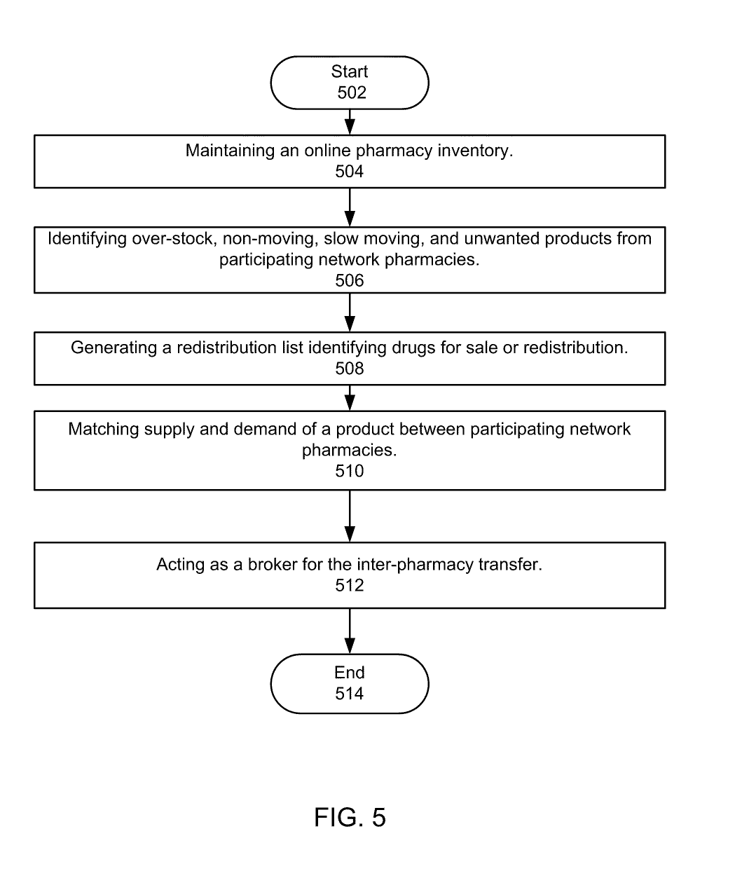

FIG. 5 illustrates a flowchart of a method to reduce product inventories, according to one embodiment of the invention. The sequence starts in operation 502. Operation 504 includes maintaining an online pharmacy inventory. Operation 506 includes automatically identifying over-stock products, non-moving products, slow moving products, and un-wanted products from the participating network pharmacies. Operation 508 includes generating a redistribution list (e.g., a list of products for resale, a list or products for redistribution, or an equivalent list). Operation 510 includes matching supply and demand between participating network pharmacies (e.g., by a computer online network, or an equivalent). Operation 512 includes acting as a broker for the inter-pharmacy transfer (e.g., of unused/overstock products). The method ends in operation 514.

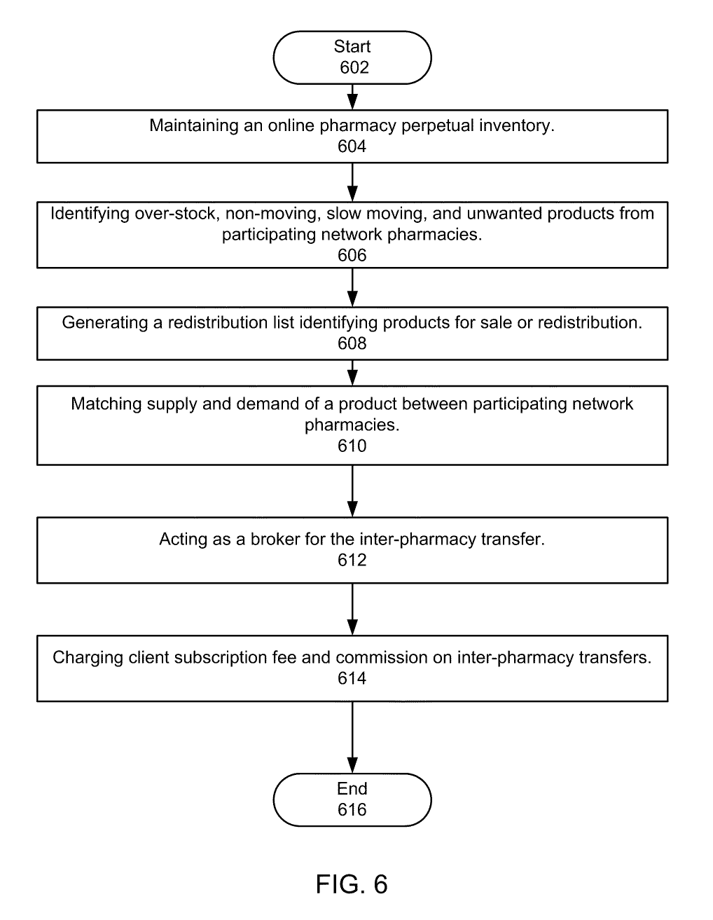

FIG. 6 illustrates a flowchart of a method to reduce product inventories, according to one embodiment of the invention. The sequence starts in operation 602. Operation 604 includes maintaining an online pharmacy perpetual inventory. Operation 606 includes automatically identifying over-stock products, non-moving products, slow moving products, and un-wanted products from the participating network pharmacies. Operation 608 includes generating a redistribution list (e.g., a list of products for resale, a list or products for redistribution, or an equivalent list). Operation 610 includes matching supply and demand between participating network pharmacies (e.g., by a computer online network, or an equivalent). Operation 612 includes acting as a broker for the inter-pharmacy transfer (e.g., of unused/overstock products). Operation 614 includes charging client subscription fees and commission on inter-pharmacy transfers. The method ends in operation 616.

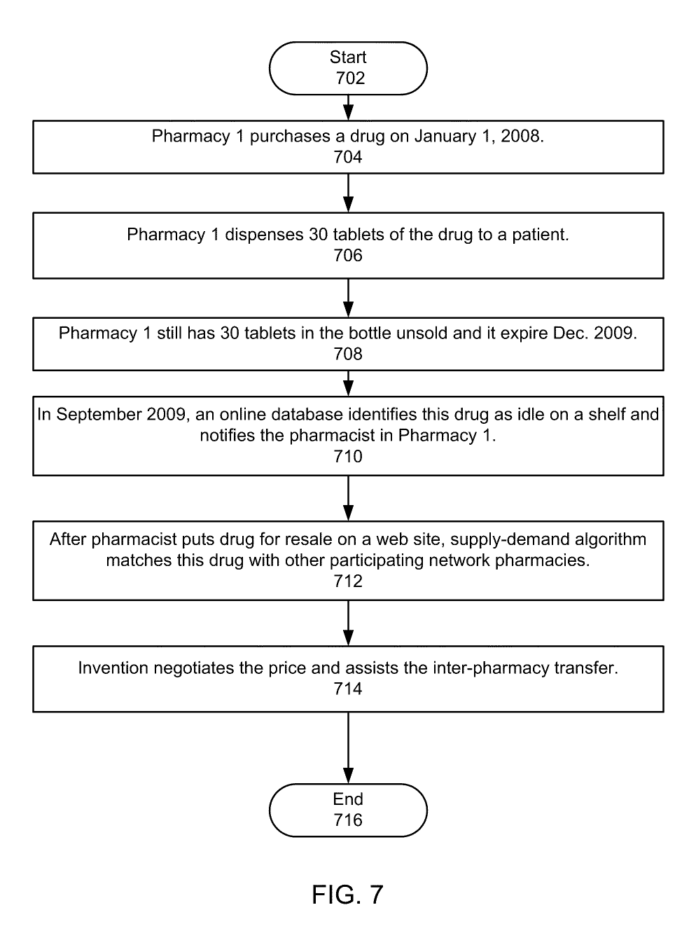

FIG. 7 illustrates a flowchart of a hypothetical and non-limiting product overstock example at a pharmacy that would typically be encountered, in accordance with one embodiment of the invention. The sequence begins in operation 702. Operation 704 includes Pharmacy 1 in Portland, Oreg., which purchased NDC #00049397060 Geodon 40 mg, 60 tablets, for $369.329 on Jan. 1, 2008. Operation 706 includes Pharmacy 1 immediately dispensing 30 tablets to one patient. Operation 708 includes Pharmacy 1 having 30 pills remaining on a shelf, and never sees another patient with similar needs. This product is expiring in December 2009. This product is non returnable and will expire if not used. Operation 710 includes in September 2009, an online database identifies this product as idle on a shelf and notifies the pharmacist. Operation 712 includes Pharmacist immediately putting this product for resale on a web-site. Through a supply-demand algorithm match with other participating network pharmacies, the invention immediately finds a match for this product. Operation 714 includes the invention negotiating the price, and assisting the inter-pharmacy transfer. The method ends in operation 716.

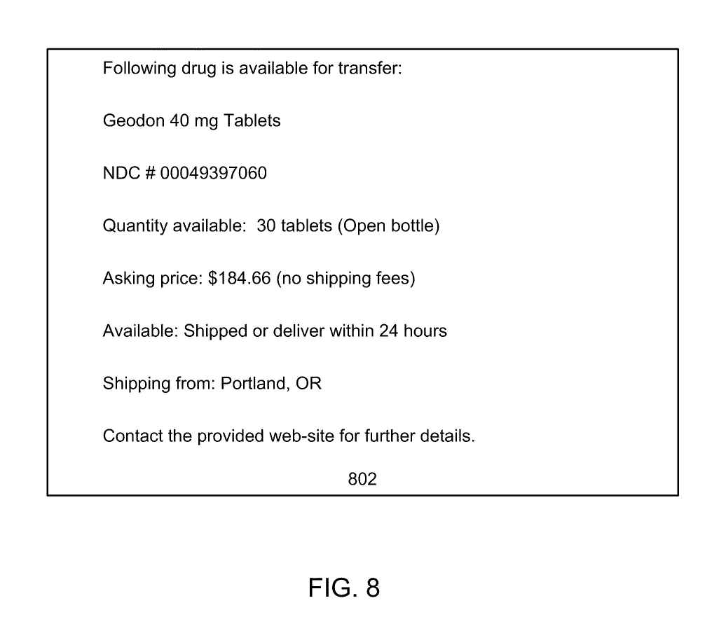

FIG. 8 illustrates a hypothetical and non-limiting example of a web-site advertisement as a result of the example in FIG. 7, in accordance with one embodiment of the invention. The advertisement 802 includes the following entries.
“Following product is available for transfer:
Geodon 40 mg Tablets
NDC # 00049397060
Quantity available: 30 tablets (Open bottle)
Asking price: $184.66 (no shipping fees)
Available: Shipped or deliver within 24 hours
Shipping from: Portland, Oreg.
Contact the provided web-site for further details.”

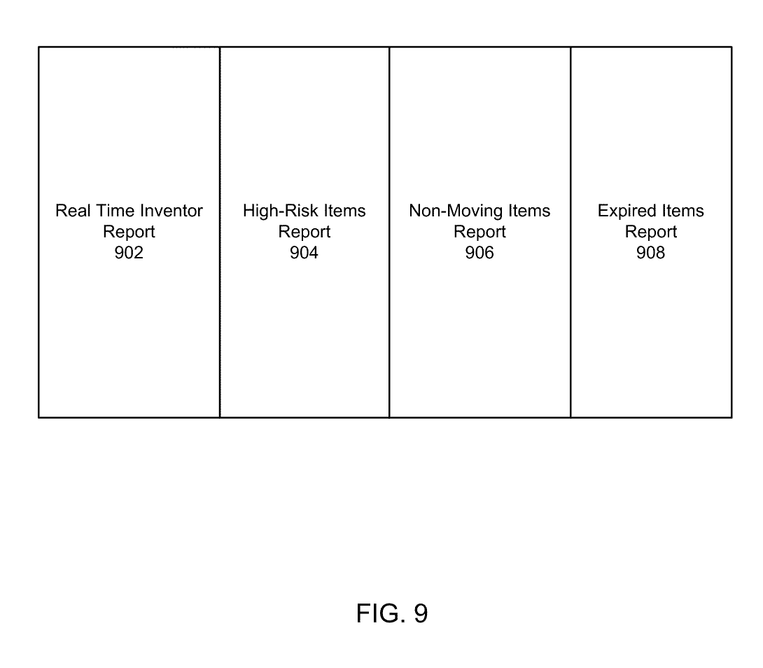

FIG. 9 illustrates a hypothetical and non-limiting example of a database, in accordance with one embodiment of the invention. There are four columns shown. The columns are Real Time Inventory Report 902, High-Risk Items Report 904, Non-Moving Report 906, and Expired Items Report 908. Real Time Inventory Report 902 lists the name of the product, the NDC, the quantity, the expiration date, the first report date, the last report date, and any notes. The High-Risk Items Report 904 lists the name of the product, the NDC, the quantity, the expiration date, and the first report date. The Non-Moving Report 906 lists the name of the product, the NDC, the quantity, the expiration date, the first report date, and the last report date. The Expired Items Report 908 lists the name of the product, the NDC, the quantity, the expiration date when the product expired, and the report date.

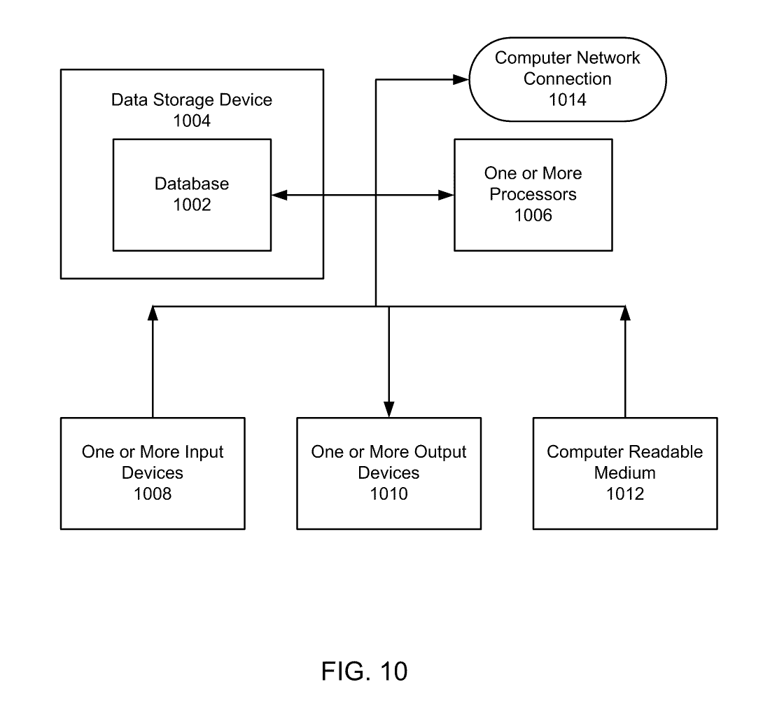

FIG. 10 illustrates a data processing system to implement the invention, in accordance with one embodiment of the invention. A Data Storage Device 1004 has a Database 1002 to hold the product information. There are one or more Processors 1006 that can access the Database 1002, as well as one or more Input Devices 1008, one or more Output Devices 1010, and a Machine-Readable Medium 1012. Also show is a Computer Network Connection 1014 to additional data processing systems (not shown). The Processors 1006 could be on one or more data processing systems (mainframe computers, client-server network of computers, desktop computers, laptop computers, palm computers, hand-held communications devices, or an equivalent). The Machine-Readable Medium 1012 could include, but is not limited to, magnetic storage medium (e.g., floppy diskette); optical storage medium (e.g., CD-ROM, DVD, or an equivalent); magneto-optical storage medium; read only memory (ROM); random access memory (RAM); erasable programmable memory (e.g., EPROM, EEPROM, or an equivalent); flash memory; electrical, optical, acoustical or other form of propagated signal (e.g., carrier waves, infrared signals, digital signals, or an equivalent); or other type of medium suitable for storing electronic instructions.

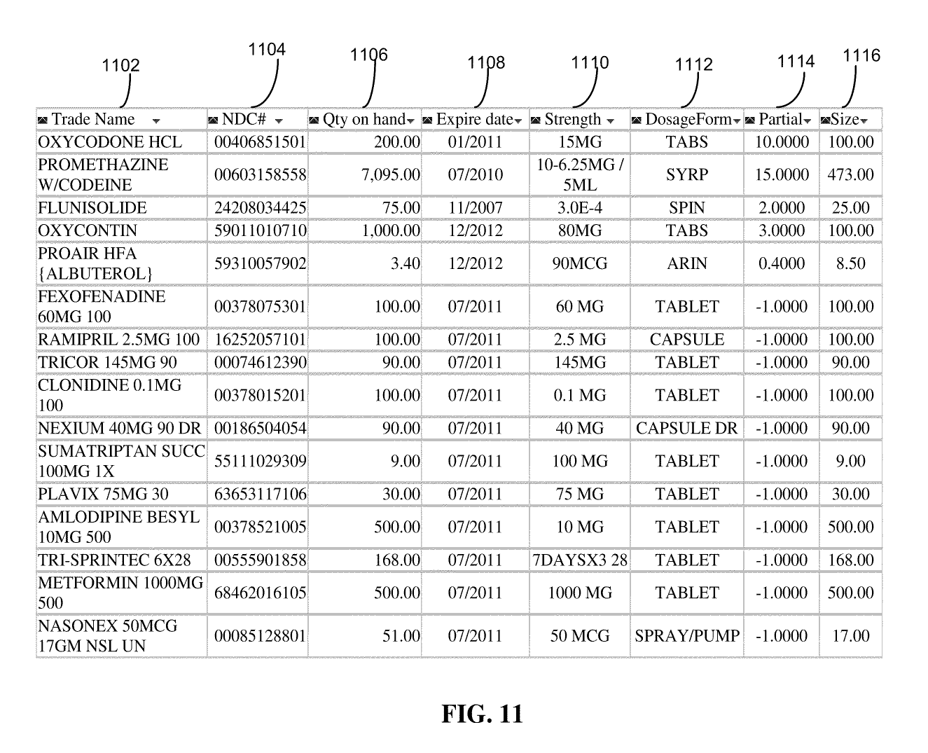

FIG. 11 illustrates a page of an inventory table of a database, in accordance with one embodiment of the invention. The first column 1102 has entries indicating the trade name of each product. The second column 1104 has entries indicating the NDC number for each product. The third column 1106 has entries indicating the quantity of each product on hand. The fourth column 1108 has entries indicating the expiration date of each product. The fifth column 1110 has entries indicating the strength of each product. The sixth column 1112 has entries indicating the dosage form (e.g., tablet, capsule, syrup, spray, and so forth) of each product. The seventh column 1114 has entries indicating any partial quantities of each product. The eighth column 1116 has entries indicating the size of each product. In alternative embodiments of the invention, the order of the columns can be ordered in a different sequence, with or without a grid. In alternative embodiments of the invention, more or less columns could be presented in this table.

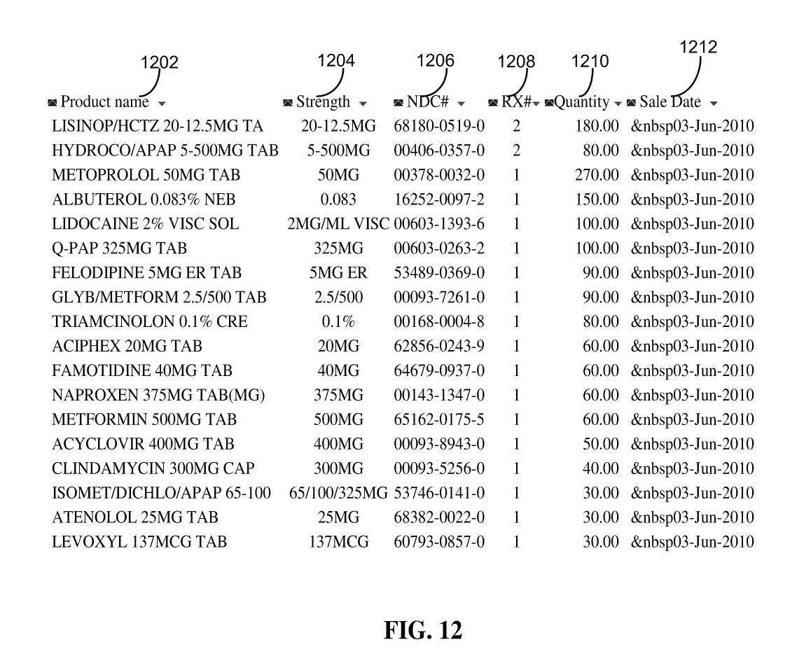

FIG. 12 illustrates a page of a sales data table of a database, in accordance with one embodiment of the invention. The first column 1202 has entries indicating the trade name of each product. The second column 1204 has entries indicating the strength of each product. The third column 1206 has entries indicating the NDC number for each product. The fourth column 1208 has entries indicating the number of prescriptions (RX) filled on a sale date. The fifth column 1210 has entries indicating the quantity of each product sold. The sixth column 1212 has entries indicating the sale date of each product. In alternative embodiments of the invention, the order of the columns can be ordered in a different sequence, with or without a grid. In alternative embodiments, more or less columns could be presented in this table.

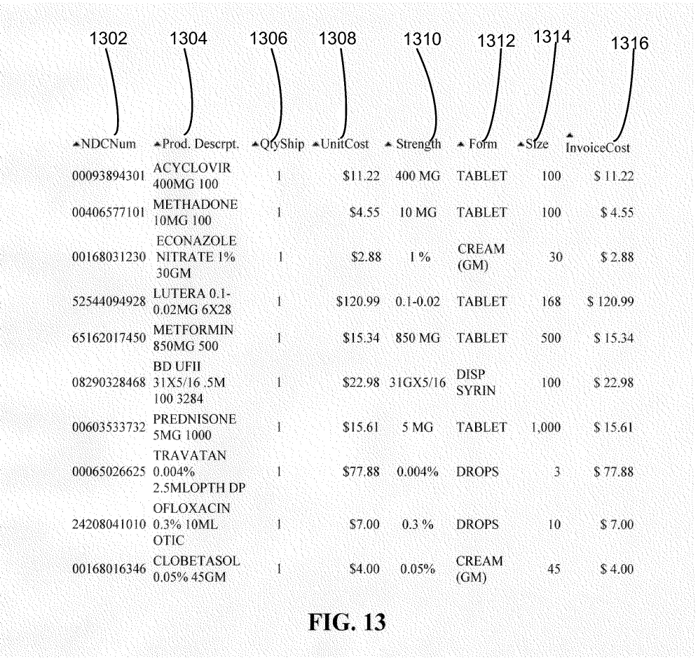

FIG. 13 illustrates a page of an invoice table of a database, in accordance with one embodiment of the invention. The first column 1302 has entries indicating the NDC number of each product. The second column 1304 has entries indicating the product description of each product. The third column 1306 has entries indicating the quantity of each product. The fourth column 1308 has entries indicating the unit cost of each product. The fifth column 1310 has entries indicating the strength of each product. The sixth column 1312 has entries indicating the dosage format (e.g., tablet, capsule, syrup, spray, cream, and so forth) of each product. The seventh column 1314 has entries indicating the size of each product. The eighth column 1316 has entries indicating the invoice cost of each product. In alternative embodiments of the invention, the order of the columns can be ordered in a different sequence, with or without a grid. In alternative embodiments, more or less columns could be presented in this table.

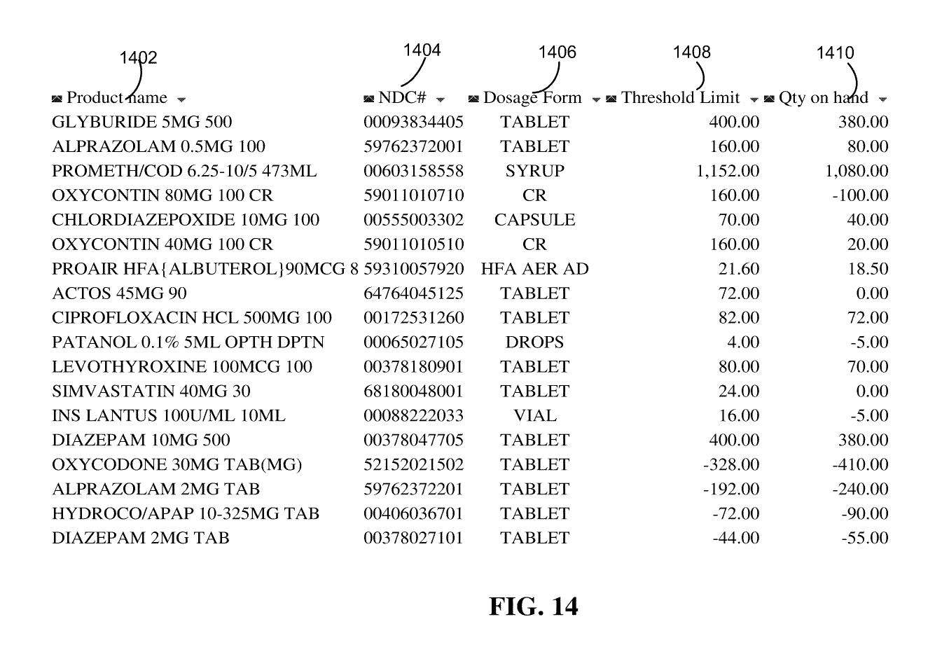

FIG. 14 illustrates a page of a threshold quantity table of a database, in accordance with one embodiment of the invention. The first column 1402 has entries indicating the trade name of each product. The second column 1404 has entries indicating the NDC number for each product. The third column 1406 has entries indicating has entries indicating the dosage format (e.g., tablet, capsule, syrup, spray, cream, and so forth) of each product. The fourth column 1408 has entries indicating the threshold limit of each product. The fifth column 1410 has entries indicating the quantity of each product on hand. In alternative embodiments of the invention, the order of the columns can be ordered in a different sequence, with or without a grid. In alternative embodiments of the invention, more or less columns could be presented in this table.

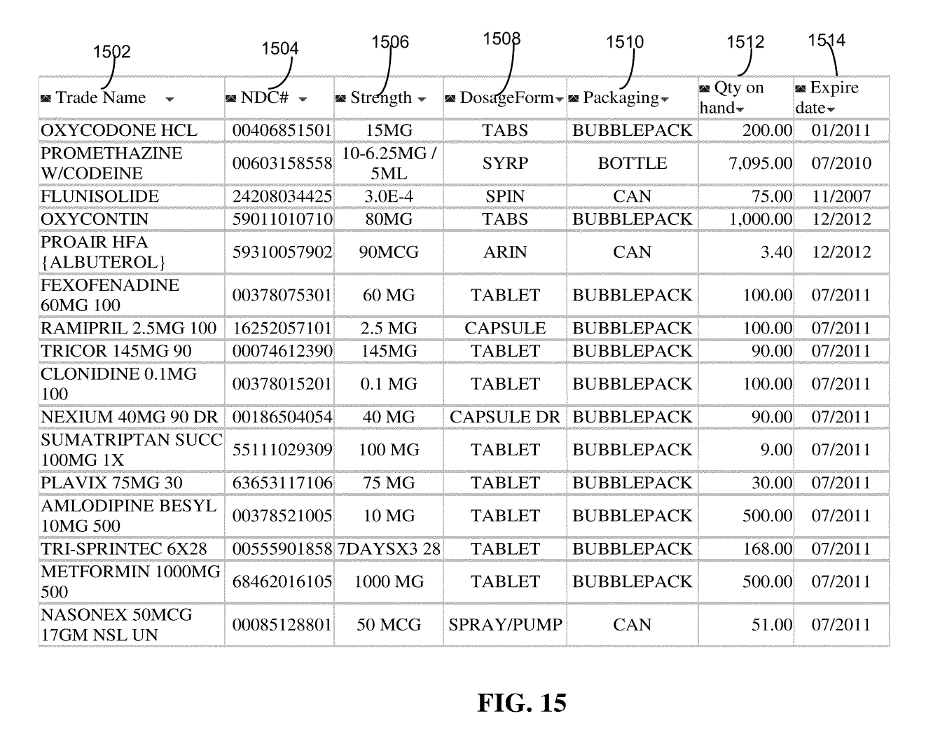

FIG. 15 illustrates a page of a product expiration table of a database, in accordance with one embodiment of the invention. The first column 1502 has entries indicating the trade name of each product. The second column 1504 has entries indicating the NDC number for each product. The third column 1506 has entries indicating the strength of each product. The fourth column 1508 has entries indicating the dosage format (e.g., tablet, capsule, syrup, spray, and so forth) of each product. The fifth column 1510 has entries indicating the packaging of each product. The sixth column 1512 has entries indicating the quantity of each product on hand. The seventh column 1514 has entries indicating the expiration date of each product. In alternative embodiments of the invention, the order of the columns can be ordered in a different sequence, with or without a grid. In alternative embodiments of the invention, more or less columns could be presented in this table.

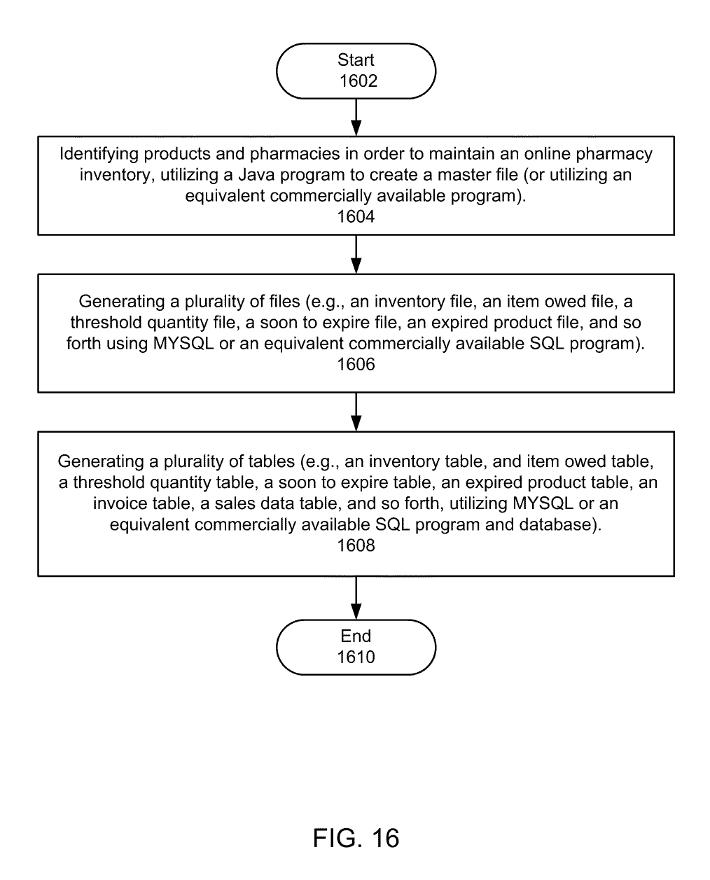

FIG. 16 illustrates a database update flowchart, in accordance with one embodiment of the invention. The sequence starts in operation 1602. Operation 1604 includes identifying products and pharmacies in order to maintain an online pharmacy inventory, utilizing a Java program to create a master file (e.g., a Java program, or an equivalent commercially available program). Operation 1606 includes generating a plurality of files (e.g., an inventory file, an item owed file, a threshold quantity file, a soon to expire file, an expired product file, and so forth using MYSQL or an equivalent commercially available SQL program). Operation 1608 includes generating a plurality of tables (e.g., an inventory table, and item owed table, a threshold quantity table, a soon to expire table, an expired product table, an invoice table, a sales data table, and so forth, utilizing MYSQL or an equivalent commercially available SQL program and database). The method ends in operation 1610.

In one embodiment of the invention, resale and redistribution can be accomplished by various services. In one embodiment, resale and redistribution occurs via physical hand to hand delivery hiring an authorized pharmaceutical delivery service. In one embodiment, resale and redistribution occurs via traditional mail delivery or courier services. In one embodiment, resale and redistribution labels are generated at a pharmacy or provided by a broker or can either be generated at a pharmacy or provided by a broker. In one embodiment, a product price of a product for resale and redistribution will be calculated based on an industry standard, such as Wholesale Acquisition Cost (WAC), Average Wholesale Price (AWP), Actual Acquisition Cost (AAC), Average Manufacturer Price (AMP), Average Selling Price (ASP), Estimated Acquisition Cost (EAC), Maximum Allowable Cost (MAC), or an equivalent.

In one embodiment of the invention, an online resale and redistribution portal will allow an option for a fixed or declined price. It will also identify full or partial quantity. If item is kept in the freezer or if it is specially handled item, the online portal will allow special notification to the seller and buyer for the specialized shipping.

In one embodiment of the invention, an online inventory database will contain the expiration dates of products, wherein the expiration date is either manually entered into the database or automatically recorded based on the default setting, or calculated based on a logic, or based on a wholesaler minimum expiration contract with the pharmacy, or based on the historical expiration date of certain class of products, or received directly from the wholesaler or a manufacture, or scanned by the pharmacy staff using hand-held scanner or custom scanner based on a manufacture provided barcode on the product. An alternative embodiment of the invention captures the expiration date and manufacturer lot number with the help of radio-frequency identification (RFID) on a product container.

In one embodiment of the invention, an online inventory system would allow operators to identify non-moving items by selecting range of dates. In one embodiment, operators can select anywhere from 1 day to 5 years in a search field. If there is no movement on that item for a selected range of time, it is identified as a non-moving item.

In one embodiment of the invention, an online inventory system would allow operators to look for their slow moving items by selecting the percentage of original item moved (i.e., sold) from the day of purchase. In one embodiment, a pharmacy operator can put anywhere from 1% to 99% in a selection criteria.

In one embodiment of the invention, an online inventory system database would allow pharmacy operators to search for items that have not expired but will expire in the future. In one embodiment, a pharmacy operator can search their inventory database to look for an item expiring within 1 day to next 5 years.

In one embodiment of the invention, an online inventory system database would allow pharmacy operators to search for items in their store inventory that have expired. In one embodiment, a pharmacy operator can search their inventory database to look for an item that expired within the previous 1 day to 5 years.

In one embodiment of the invention, an online inventory system database would allow pharmacy operators to search for top selling items in their store inventory. In one embodiment, a pharmacy operator can search their inventory database to look for an item that is a top selling item on a daily basis, weekly basis, monthly basis, or yearly basis, either counted by prescriptions filled or number of units sold.

In one embodiment of the invention, an online inventory system database would allow pharmacy operators to identify and return unused or unopened items back to a wholesaler for credit. This query is based on a client contract with a wholesaler allowing a “Return to wholesaler” option.

In one embodiment of the invention, an online inventory system database would allow pharmacy operators to set-up a threshold limit for each item in the store. If an item count goes below certain limit, a pharmacy operator will get a threshold limit notification, where the item will be highlighted in red, or an equivalent notification will be used.

In one embodiment of the invention, an online inventory system database can record a plurality of items that have expiration dates. In one embodiment, an item is counted on a day of physical inventory, where an item counted after physical inventory automatically receives a default expiration of 6 months, 9 months, or 12 months, depending on a client contract with a wholesaler.

In one embodiment of the invention, an online inventory system database would allow pharmacy operators to link to a distributor database to view a return policy and manage the product accordingly. In one embodiment of the invention, an online inventory system database would allow pharmacy operators to see dollar values for each item on hand along with the total dollar amount of the inventory at any given time. In one embodiment of the invention, an online inventory system database would allow pharmacy operators to create a custom report online or print out an automatically generated report at any given time. In one embodiment of the invention, the custom reports can include one or more of the following: a list the current store inventory, a list of non-moving items, a list of slow-moving items, a list of expired products, a list of products nearing expiration, a list of top selling items, a list of lowest selling items, and a list of percentage of particular product moved. In one embodiment, custom reports can be set-up to automatically be provided at a specified interval. The database can be physically verified by visits to a pharmacy every few months to verify the accuracy of the inventory and expiration database, either manually, or with the use of hand-held device.

Returning and/or redistributing unused and overstocked products would allow the dead stock to be unfrozen and make more cash available for a pharmacy. When one pharmacy is returning an expensive expired prescription product from their store, some other pharmacy near or far is ordering the same product from the wholesaler. By identifying these matches, the pharmacies would be able to significantly reduce their loss of expired products and prevent over production of pharmaceuticals, and reduce the chemical waste in the environment. Pharmacy owners of more than one pharmacy and also chain stores can save significant money by transferring unused products between their stores.

In one embodiment of the invention, an online inventory system database would allow pharmacy operators to use a National Product Code (NDC) product master file as a reference or as a skeleton for identifying a product transaction. A NDC database can be embedded as a skeleton for the online inventory database and can be frequently up-dated. This database can be acquired directly through the Food and Drug Administration (FDA), or acquired from private vendors (e.g., First Databank, Medi-Span, Wolters Kluwer Health, the CommonDataHub repository, the National Institute of Health, or an equivalent source). One embodiment of the invention can utilize the most current NDC master file to help pharmacy operators identify recalled product products and identify obsolete NDCs from their pharmacy computer.

In one embodiment of the invention, an online inventory system database will allow formation of a “Repository Pharmacy.” This type of pharmacy would only carry non-common items, hard to sale items, items with odd packaging size, frequently expired products, items with very short expiration dates. Participating pharmacies can thereby stop ordering the items already stocked in the Repository Pharmacy. These medicines can be transferred immediately to the participating pharmacies if a prescription arrives. This concept will avoid any unnecessary stockpiling of non-moving items in multiple pharmacies. A Repository Pharmacy to carry hard to sell products, non-moving products, newly launched products, and oddly packaged quantities of products can save money for pharmacy operators. At any given time if medications from repository pharmacies are expired, all the participating pharmacies will share the burden and can tremendously reduce the cost of their inventory and ultimately redistribution. Repository pharmacies can be strategically located to provide more timely delivery of a medication.

In one embodiment, the resale and redistribution processes can be implemented using different methods. For example, resale and redistribution processes could be implemented using an open source resale network, a closed source resale network, and/or a comprehensive resale network.

In one embodiment, an open source resale network can enable pharmacies with valid DEA number to register and buy or sale unwanted/overstock prescription products. An open source resale network can provide the largest exposure to a seller and buyer because all pharmacies with a valid DEA number could be a participant in the open source resale network. For example, independent pharmacy owners with a single pharmacy could resell or redistribute their products using the open source resale network. The open source resale network allows independent pharmacies to liquidate/resale/transfer their idle stock to other pharmacies or purchasers.

In one embodiment, a closed source resale network can be available to pharmacies with multiple pharmacy operators and/or commonly control pharmacies. Chain drug stores, multi-location hospital pharmacies and multiple independent pharmacy owners could sell or transfer prescription products between their commonly controlled pharmacies using the various aspect methods and systems. Prescription products sold or transferred on the closed source resale network would typically not be open to other not commonly owned or operated pharmacies. The closed source resale network enables each pharmacy client with multiple pharmacy locations to manage and monitor only their own inter-store transactions.

In one embodiment, a comprehensive resale network can provide users with an inventory management and monitoring system in addition to enabling the clients to use the open source and close source resale networks. The comprehensive resale network would not differentiate between a single pharmacy owner versus multi-pharmacy owners.

In one embodiment, different methods and systems can be employed to facilitate the sale of products. For example, products can be sold from one pharmacy to another through direct transaction between the pharmacies, through a third party vendor, such as PayPal®, or by bidding. Various embodiments can enable pharmacies to sell/purchase products to/from other entities, reverse distribute products for credit, transfer products to or receive products from another affiliated entity, exchange products with other entities using barter principles or donate products to other entities.

Various embodiments for online inventory systems and methods are possible. For example, various systems and methods can be useful in the shelf-life monitoring, inventory monitoring, inventory management, and supply chain management in any industry. Any product that has an expiration date could be monitored and managed using the various embodiments of the invention. Pharmaceutical products, chemicals, biotechnology products, natural products, synthetic products, semi-synthetic products, infant formulas, baby products, nutritional supplements, vitamins, meat and dairy products, canned foods, condoms, car seats, make-up and skin-care products, beauty products, hair products, and homeopathic products are just a few examples of products and industries that can use various embodiments of the invention. In particular, various embodiments could be useful in tracking the expiration date of healthcare products.

Several embodiments of the invention are possible. The phrase “in one embodiment” used in the specification can refer to a new embodiment, a different embodiment disclosed elsewhere in the application, or the same embodiment disclosed earlier in the application. The exemplary embodiments described herein are for purposes of illustration and are not intended to be limiting. Therefore, those skilled in the art will recognize that other embodiments could be practiced without departing from the scope and spirit of the claims set forth below.

What is claimed is:

1. A method to electronically manage pharmacy drug inventory including near expiry drug products, said method comprising:
(a) electronically maintaining an online drug inventory among a plurality of participating network pharmacies or related vendors by using one or more specifically programmed data processing systems,
(b) electronically identifying and tracking drug products selected from near expiry drug products and at least one selected from over-stock drug products, non-moving drug products, slow moving drug products, and expired drug products from two or more of the plurality of participating network pharmacies or related vendors,
(c) electronically generating redistribution lists of one or more of said corresponding drug products identified in step (b), and
(d) redistribution or transferring using the redistribution list one or more of the drug products for disposal, supply and demand between two or more of the plurality of participating network pharmacies or related vendors.

2. The method of claim 1, further comprising:
providing brokering, facilitating, or wholesaling of the transfer of drug products between the plurality of participating network pharmacies or related vendors.

3. The method of claim 1, further comprising:
charging a fee or commission for accessing the online pharmacy inventory.

4. The method of claim 1, further comprising:
charging a client subscription fee for accessing the online pharmacy inventory or a commission on at least one transfer or redistribution.

5. The method of claim 1, wherein the generating of the redistribution list of one or more products includes one or more of:
(a) generating a list of one or more products for resale and redistribution; or
(b) utilizing a computer online network.

6. (canceled)

7. (canceled)

8. A method to electronically manage pharmacy drug inventory, said method comprising:
(a) electronically maintaining an online pharmacy inventory among a plurality of participating network pharmacies or related vendors,
(b) electronically automatically identifying and tracking drug products selected from near expiry drug products and at least one of over-stock drug products, non-moving drug products, slow moving drug products, and expired drug products from the plurality of participating network pharmacies or related vendors,
(c) electronically generating one or more redistribution lists of one or more of said drug products for resale, redistribution, or disposal,
(d) redistributing or transferring of the drug products, using one or more of the redistribution lists, for disposal, supply and demand between two or more of the participating network pharmacies or related vendors by using one or more specifically programmed data processing systems, and
(f) charging a fee or a commission on at least one of said redistributions or transfers.

9. The method of claim 8, wherein generating a redistribution list of one or more drug products includes generating a list of one or more drug products for resale and redistribution using one or more reports or one or more alerts generated through a drug product inventory management module or generated from input provided by one or more participating pharmacies or related vendors.

10. (canceled)

11. The method of claim 8, wherein the method includes transfer of unused drug products and overstock drug products.

12. The method of claim 8, further comprising:
brokering, facilitating, or wholesaling transfer of one or more drug products without taking possession of the one or more products.

13. The method of claim 8, further comprising:
brokering, facilitating, or wholesaling transfer of one or more drug products by buying the one or more drug products from the plurality of the pharmacies or related vendors and storing the one or more drug products in a warehouse for resale.

14. The method of claim 8, further comprising:
viewing drug product inventory or the redistribution list from any computer with Internet access.

15. The method of claim 8, wherein an online pharmacy inventory database comprising the drug product inventory or the redistribution list also serves as a data repository for one or more selected from: wholesaler invoices, daily sales reports, reverse distributor statements, pharmacy registrations and DEA numbers, and account holder names.

16. A system to electronically manage pharmacy drug inventory, said system comprising:
(a) an electronic data processing system network to maintain an online pharmacy inventory among a plurality of participating network pharmacies or related vendors, using one or more specifically programmed data processing systems,
(b) an electronic database on the data processing system to identify near expiry drug products and at least one of expired drug products, over-stock drug products, non-moving drug products, slow moving drug products, and un-wanted drug products from the plurality of participating network pharmacies or related vendors,
(c) an electronic program module on the data processing system to generate a redistribution list of one or more drug products, and
(d) an electronic program module to redistribute or transfer one or more of said drug products for disposal, supply, and demand between two or more of the plurality of participating network pharmacies or related vendors.

17. (canceled)

18. The system of claim 16, wherein redistribution or transfer utilizes one or more of the following types of delivery: physical hand-to-hand delivery hiring an authorized pharmaceutical delivery service, mail delivery, and courier services.

19. The system of claim 16, wherein a drug product sales report is electronically provided to a pharmacy or related vendor by utilizing a switch provider.

20. The system of claim 16, wherein resale and redistribution labels are generated at a pharmacy, related vendor, or provided by a broker.

21. The system of claim 16, wherein a product price of a drug product for redistribution or transfer is calculated based on an industry standard chosen from the list of industry standards consisting of: Wholesale Acquisition Cost, Average Wholesale Price, Actual Acquisition Cost, Average Manufacturer Price, Average Selling Price, Estimated Acquisition Cost, and Maximum Allowable Cost.

22. The system of claim 16, further comprising:
an online inventory database which contains a plurality of expiration dates corresponding to a plurality of drug products, wherein a plurality of expiration dates can be entered by one or more sources, consisting of one or more selected from:
manual entry into the database, automatic entry in the database based on a default setting, automatic entry in the database based on a wholesaler minimum expiration contract, automatic entry in the database based on a historical expiration date of a certain class of products, automatic entry in the database based on dates directly received from a wholesaler or manufacturer, and entry in the database from scanning a barcode on a drug product.

23. (canceled)

24. The system of claim 16, wherein an online inventory system allows an operator to perform one or more of the following:
(a) search for drug products that have not expired,
(b) search for drug products that have expired,
(c) to look for slow moving drug products by selecting the percentage of original drug products moved from the day of purchase;
(d) search for drug products that are top selling;
(e) search for drug products to identify and return if unused to a wholesaler for credit;
(f) set-up a threshold limit for one or more drug products in a store, wherein if a drug product count goes below a predetermined limit, the operator receives a threshold limit notification; or
(g) use a National Drug Code master file for one or more drug products in a store to identify one or more recalled drug products.

25. (canceled)

26. (canceled)

27. (canceled)

28. (canceled)

29. (canceled)

30. The system of claim 16, wherein an online inventory system supports a repository pharmacy that carries a plurality of one or more: hard to sell drug products, drug products with odd packaging sizes, frequently expired drug products, and drug products with short expiration dates that are transferred to a plurality of pharmacies or vendors as needed.
